(19) United States
(12) Patent Application Publication
(10) Pub. No.: US 2008/0140261 A1
(43) Pub. Date: Jun. 12, 2008

(54) METHOD AND APPARATUS FOR MIXING TWO OR MORE GASEOUS OR LIQUID STREAMS

(76) Inventors: Tommy Hansen, Tikob (DK); Martin Skov Skjoth-Rasmussen, Kokkedal (DK)

Correspondence Address: DICKSTEIN SHAPIRO LLP, 1825 EYE STREET NW, Washington, DC, 20006-5403, US

(21) Appl. No.: 11/939,428

(22) Filed: Nov. 13, 2007

(30) Foreign Application Priority Data
Dec. 9, 2006 (DK) PA 2006 01621

(51) Int. Cl. G05D 7/00; C07C 2/82

(52) U.S. Cl. 700/282; 137/3; 518/703

(57) ABSTRACT

The invention provides a method for mixing two or more gaseous or liquid streams, where the gaseous streams are combined in an inlet chamber and thereafter repeatedly accelerated and decelerated in one or more of stages, and a value of a maximum linear velocity of the accelerated combined gaseous stream is maintained in each step within a range of mass flow rates of the gaseous streams.

The invention also provides apparatus for mixing two or more gaseous or liquid streams, one embodiment comprises a body with a seat; a spindle with a plug, which is installed in the seat and the seat and the plug have a plurality of conical surfaces forming the same plurality of conical annuli. The spindle is able to move the plug in an axial direction in the seat during the mixing.

Another embodiment comprises a body, a spindle with a tube plug. A cage and annular mixing elements surround the tube plug.

### BACKGROUND OF THE INVENTION

1. Field of the Invention

The invention relates to thorough mixing of two or more fluid streams.

The invention is specifically directed to a method and an apparatus for mixing two or more fluid streams, where variations in the flow rates occur.

The invention is especially useful in catalytic partial oxidation reactors, where hydrocarbon feed and oxidant feed must be very thoroughly mixed. This is important for obtaining optimal reaction in a subsequent catalyst bed.

2. Description of related Art

Fluid mixers are known in the art, also those for mixing a hydrocarbon with an oxidant prior to a catalytic partial oxidation.

For this purpose Schulzer Chemtech has developed a static mixer, which they show in a brochure available on the internet. The mixer comprises a tube with blades on the inner surface or on a shaft installed in the tube and the blades create a mixing, turbulent flow pattern.

In U.S. Pat. No. 5,026,946 a mixer is shown, where a hydrocarbon is mixed with an oxidant. The mixer comprises two concentric tubes; the inner tube is closed in one end and is equipped with small holes. The hydrocarbon flows in the annular space between the tubes, and the oxidant flows from the inner tube out through the holes and is mixed with the hydrocarbon.

U.S. Pat. No. 5,112,527 discloses a process for autothermal reforming of lower alkanes such as natural gas. In order to homogeneously blend the gaseous alkanes, steam and oxygen containing gas a static mixer is installed in an inlet channel. Efficiency of mixing and created pressure drop in the static mixer, however, will vary with the amount of gas flowing through the static mixer.

Another mixer is described by JP 3213132, where the gases are mixed by a rotating shaft in a housing. The surface of the shaft and inner surface of the housing both are in a shape of a screw groove. This pushes the gases forward in a flow passage with flow areas having a certain maximum and minimum size.

A mixer/diffuser disclosed in U.S. Pat. No. 6,092,921 comprises an inlet chamber, an expander and an outlet chamber, where a body is inserted in the expander creating a conical, annular flow passage.

Thorough mixing of two or more gases or liquids inevitably costs pressure drop. Common to the mixing devices of prior art is that, when flow rate increases during operation, the created pressure drop increases considerably. And when the flows decrease the mixing quality decreases as well.

It is therefore the object of the present invention to provide a simple method and apparatus for mixing, which thoroughly mix two or more fluids, and equally thoroughly at varying flow rates, but without variations in the created pressure drop across the mixer and in mixing efficiency.

### SUMMARY OF THE INVENTION

Pursuant to the above object the invention relates to a method for mixing two or more fluid streams, where the streams are combined in an inlet chamber and thereafter repeatedly accelerated and decelerated in one or more stages. A value of a maximum linear velocity of the accelerated combined streams is maintained in each step within a range of mass flow rates of the feed streams by adjusting area of smallest flow passage.

The invention also provides apparatus for mixing two or more gaseous or liquid streams. One embodiment of the apparatus comprises a body with a seat; a spindle with a plug, which is installed in the seat and the seat and the plug have a plurality of conical surfaces forming the same plurality of conical annuli. The spindle is able to move the plug in an axial direction in the seat during the mixing operation. The spindle and the plug may comprise a flow passage.

Another embodiment of the invention provides an apparatus for mixing two or more gaseous or liquid feed streams and comprises a body and a movable spindle connected to a tube plug coaxially installed in the body. Near the spindle the tube plug is perforated, and it is open in the other end. The feed streams enter the tube plug through the perforation holes. A cage with nozzles surrounds the tube plug, a plurality of annular mixing elements surround the cage, and the mixing elements are parted from each other. The cage and the mixing elements are closed at the end, where the tube plug end is open.

The invention ensures thorough mixing of fluids at a constant pressure drop for a range of flow rates.

### BRIEF DESCRIPTION OF THE DRAWINGS

FIG. 1 is a cross-section of one embodiment of the mixing device of the invention.

FIG. 2 is a cross-section of a mixing device of the invention installed at the inlet of a reactor.

FIG. 3 is a diagram of a reactor with a feed gas mixed and controlled according to the invention.

FIG. 4 is a cross-section of another embodiment of the mixing device of the invention.

FIG. 5 is a cross-section of yet another embodiment of the mixing device of the invention.

### DETAILED DESCRIPTION OF THE INVENTION

Mixing of gasses and/or liquids takes place in all processes, and performance of equipment installed downstream of such mixing is dependent of efficiency of the mixing.

One example is H₂/CO containing synthesis gas production from a hydrocarbon, steam and an oxidant, which can be air, oxygen or a mixture, i.e. enriched air. The raw materials are mixed and thereafter catalytically oxidised. Further, it is important to avoid reaction between oxygen and hydrocarbon before the gas mixture enters a catalyst bed.

To obtain an optimal reaction in the catalyst bed, it is very important that the gas streams are thoroughly mixed before entering the catalyst bed.

An efficient way of mixing two or more gases or liquids is to let the streams, which just are led together, accelerate and decelerate a number of times and with a sufficient high maximum velocity, which helps to prevent reaction upstream of the catalyst bed. When the accelerated streams are decelerated in a subsequent chamber, eddies are created in the chamber and the streams are mixed.

This pressure drop is rather high compared to pressure drops created by other piping elements and reactor internals in petrochemical plants.

It is important to obtain a thorough mixing at all capacities of a plant without creating excessive pressure drops at high capacities, and without loosing mixing efficiency at low capacities of a plant.

The invention provides a method and an apparatus for mixing two or more fluids by repeated acceleration and deceleration and at a constant pressure drop for a wide range of capacities. This is obtained by changing the flow area of the narrow flow passage in the acceleration part of the mixer. In this way, the fast linear velocity of the fluid, the created pressure drop and the mixing efficiency are maintained constant for a wide range of flow rates.

It has now been found that this is obtained by a mixer comprising a seat and a spindle with a plug, where the surface of the seat and of the plug form annuli and chambers, through which the fluids flow. The annuli are conical, which enables the spindle to be moved up and down (when vertically installed) resulting in adjusted flow areas.

The seat comprises holes between chambers, which are bore flow connections between chambers. This forces the gases in the chambers to change flow direction 90° a number of times, typically 3-5 times, depending of the specific design.

The spindle and the plug can be bored, so a liquid can flow in this passage and out through a spray nozzle mounted on the plug.

Alternatively, the movable plug can be a tube, with holes in the end at the spindle end and the other end being open. Instead of a seat, the tube plug is surrounded by a cage, which is surrounded by horizontal gas mixing elements. The elements are separated from each other, and thereby the feed gasses only flow through the elements and the part of the cage, which are not blinded off by the tube plug.

This keeps the maximum linear velocity and thereby also the desired pressure drop and the eddy formation constant, which is important for good mixing of streams with varying flow rates.

A mixer will be designed to obtain the best mixing at a certain pressure drop, which then has to be kept constant.

When designing a gas-gas mixer with annular flow passage between seat and plug, the relation between flow rates and flow area of an annulus is expressed as

$${R}_{1} = \frac{F}{{D}_{seat}^{2} - {D}_{plug}^{2}} * \frac{{P}_{ref}}{P} * \frac{T}{{T}_{ref}}$$

where

F is total gas flow rate in Nm³/sec

Dₛₑₐₜ and D_{plug} are inner diameter of seat and diameter of plug at the same position in an annulus in m.

P is pressure in MPa in the mixer and P_{ref} is 3.0 MPa,

T is temperature in K in the mixer and T_{ref} is 473.15 K.

R₁ must range between 1*10⁶ and 1*10⁸ Nm³/sec/m² preferably between 5*10⁶ and 2*10⁷ Nm³/sec/m² .

The relation between the combined feed flow rate and the cross sectional area of the holes between the chambers in the seat is expressed by ratio R₂ which can be expressed as

$${R}_{2} = \frac{F}{n * {D}_{hole}^{2}} * \frac{{P}_{ref}}{P} * \frac{T}{{T}_{ref}}$$

where

F is total gas flow rate in Nm³/sec,

n is number of holes,

Dₕₒₗₑ is diameter of one hole between chambers in m,

P is pressure in MPa in the mixer and P_{ref} is 3.0 MPa,

T is temperature in K in the mixer and T_{ref} is 473.15 K.

R₂ must range between 5*10⁵ and 1*10⁷ Nm³/sec/m² preferably 1*10⁶ and 2*10⁶ Nm³/sec/m².

The mixer can be installed anywhere a thorough mixing of two or more fluids is required. In a CPO reactor it is convenient to install the mixer in an inlet flange of the reactor. The mixer is further described by the drawings.

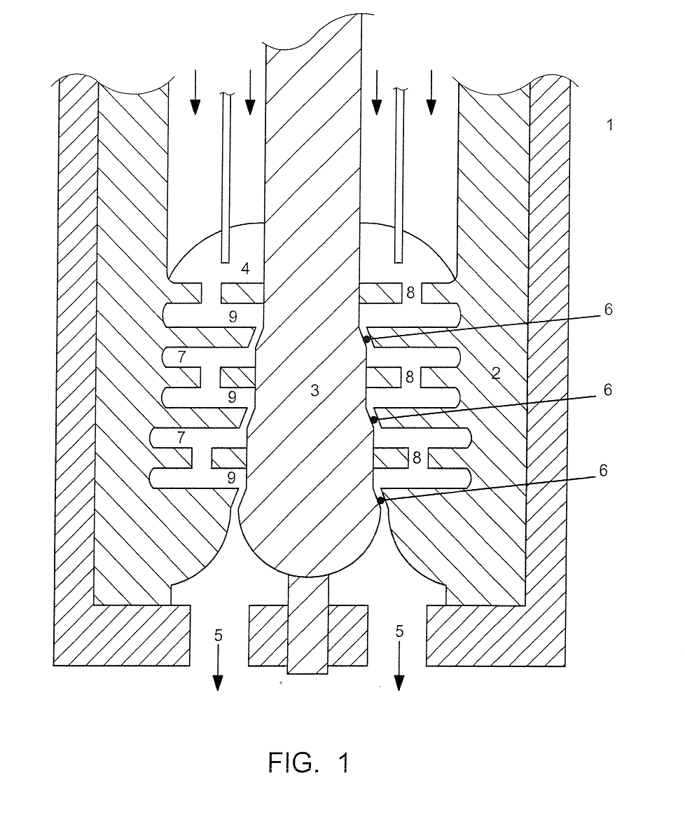

One embodiment of the invention is shown by FIG. 1. Mixer 1 comprises a seat 2 and a plug 3; the fluids to be mixed enter through two annular channels as indicated by arrows, from where they flow to an inlet chamber, which optionally is filled by a porous medium 4, from where they flow through holes 8 to chamber 9. The mixed fluids leave through outlets 5. After having entered the chamber 9, the fluids flow through the first of the annuli 6, where the fluids accelerate between the plug 3 and the seat 2. From the annulus the fluids flow with high velocity out into one of chambers 7, where they are decelerated and forced to change directions, first 90° from vertical to horizontal into the chamber, and then from horizontal to vertical to pass through holes 8 to chamber 9. In chamber 9 eddies are formed resulting in additional, thorough mixing. Then the direction changes from vertical to horizontal and then again to vertical for flowing into a subsequent annulus 6. Thereby, turbulence and proper mixing is created in the chambers 7 and 9.

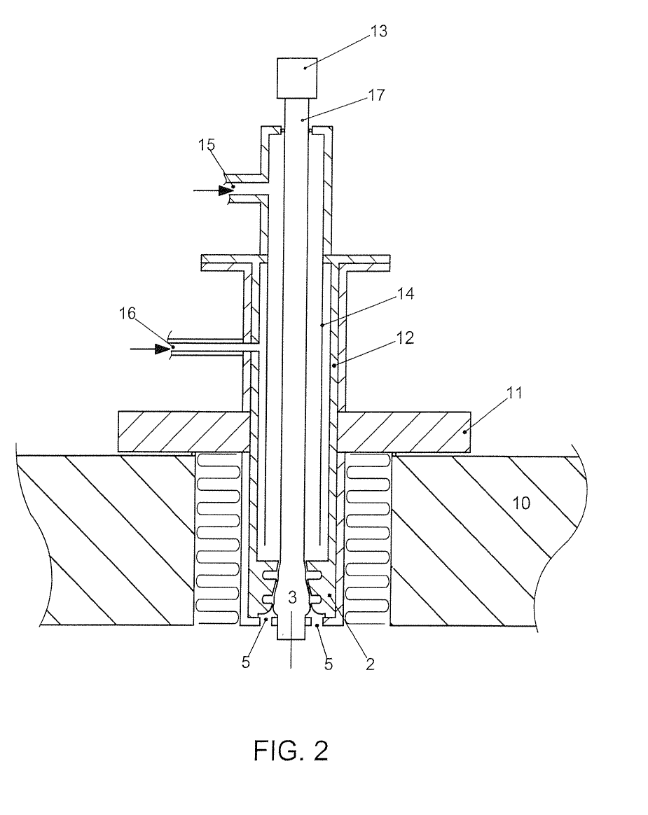

Installation of one embodiment of the mixer is shown on FIG. 2. The mixer is installed at a top inlet 10 of a catalytic partial oxidation, CPO, reactor, to which a body 11 is connected. In the body 11 a guide 12 is inserted and a spindle 17 of the plug 3 runs through the body 11 and the guide 12 and can be moved up and down by actuator 13, which thereby alters flow area between the seat 2 and the plug 3. Between the spindle 17 and the guide 12 an inner tube 14 is installed.

The oxidant/steam feed enters the mixer through oxidant inlet 15 and flows between the spindle 17 and the inner tube 14, while the hydrocarbon/steam feed enters through hydrocarbon inlet 16 and flow between the inner tube 14 and the guide 12. These gases flow together at the acceleration/deceleration part of the mixer, downstream of which the mixed gas leaves the mixer through outlet 5 and enters the CPO reactor.

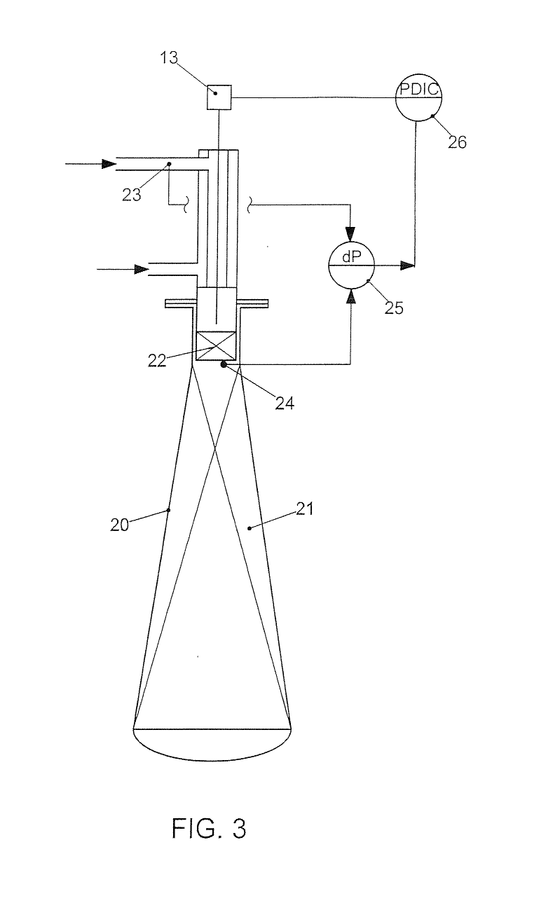

The performance of the mixer during operation is shown on FIG. 3. In the CPO reactor 20 catalyst bed 21 is installed. The mixed gas flows through flame arrestor 22 to the space in the CPO reactor inlet of the catalyst bed 21.

A pressure gauge 23 is installed at the oxidant inlet pipe and another pressure gauge 24 at the outlet of the mixer. The signals from 23 and 24 are received by pressure measuring instrument 25, which calculates the pressure drop across the mixer and sends this signal to controller 26. Controller 26 keeps the pressure drop constant by sending a signal to actuator 13, as the actuator moves the spindle up or down adjusting the flow area of the annuli. This ensures the constant pressure difference and the optimal mixing at a wide range of operating capacities.

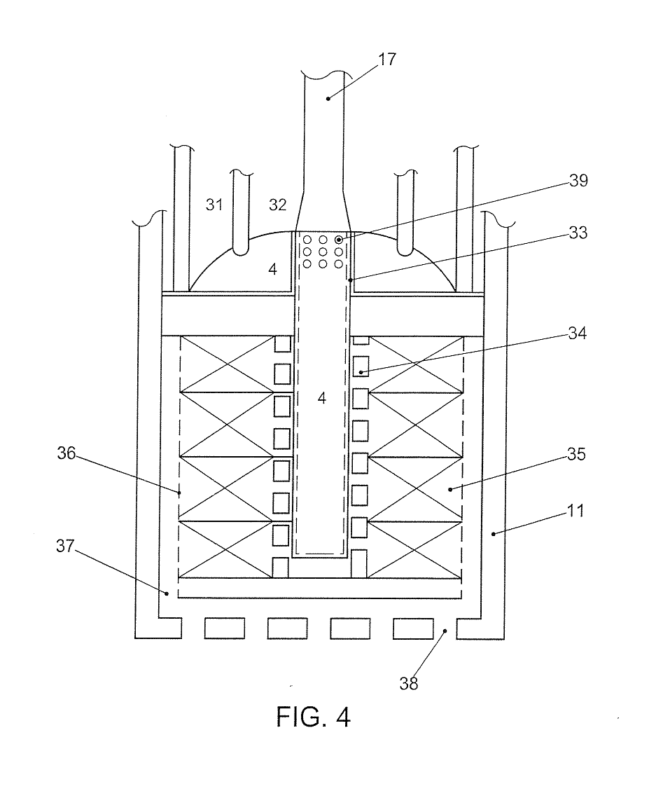

In FIG. 4 another embodiment of mixer is shown. In this, the mixer 1 comprises body 11 and spindle 17, which is connected to a tube plug 33. Near the spindle 17 the tube plug 33 is equipped with holes 39, at the other end the tube plug 33 is open. Optionally, upstream of the holes 39 and inside the tube plug 33 a porous material 4 is installed. The tube plug 33 is coaxially surrounded by a cage 34, i.e. a tube with nozzles. The space between tube plug and cage is just so wide that the tube plug can slide in the cage. The nozzles can be arranged in a pre-determined pattern, such as a helical pattern. Around cage 34, horizontal, annular wire mesh mixing elements 35 are placed, which thereby are horizontally isolated from each other. The elements 35 are surrounded by a perforated tube 36.

The cage and the mixing elements are closed in the lower end where the tube plug is open. The height of the cage 34—when vertically installed—and the height of non-perforated part of the tube plug 33 are substantially the same. Thereby, the non-perforated part of the tube plug 33 is able to block off zero, some or all of the nozzles, when positioned in the upper, a middle or the lower position, respectively.

Further, the total area of the cage nozzles is considerably smaller than flow area of any other flow passage of the mixer.

The oxidant/steam inlet stream enters the mixer from inlet annulus 32 and the hydrocarbon/steam inlet stream enters from the surrounding inlet annulus 31. Both streams flow into a porous material 4, from where they enter the tube plug 33 through the inlet holes 39. After the gas streams are mixed, the mixed gas flows from the perforated tube 36 into an outlet channel 37 and leaves the mixer 1 through outlet holes 38.

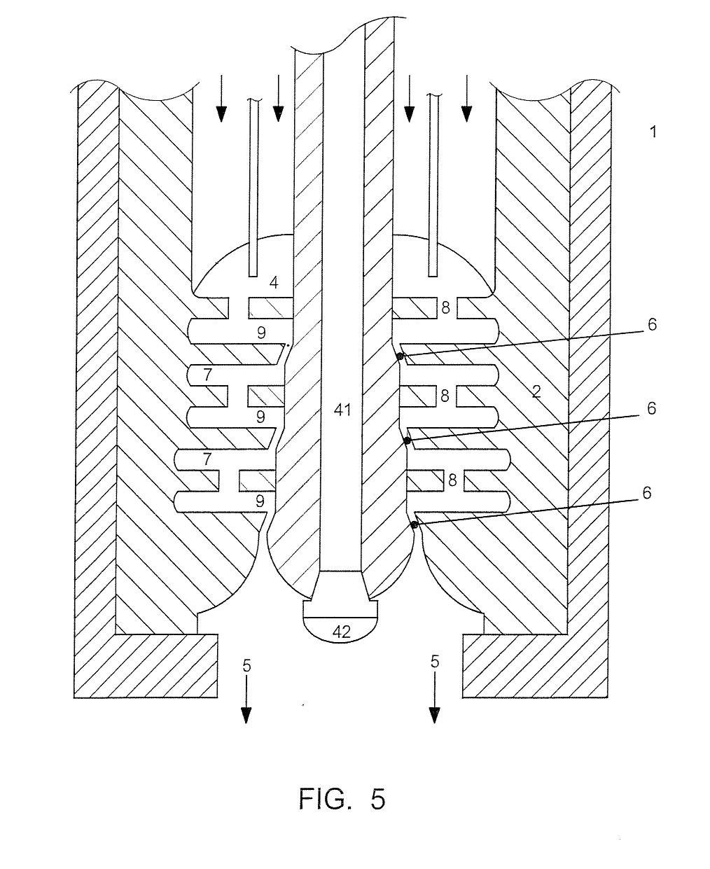

A further use and embodiment of the mixer with annular gas flow passages is shown in FIG. 5. In this embodiment a flow channel 41 is bored in the spindle and the plug 3, and a liquid can thereby flow in this internal liquid flow passage. At the outlet of the liquid flow passage a spray nozzle 42 is connected to the plug 3, so a spray of liquid is introduced with high velocity into the mixed gas. An example of liquid in the channel is a liquid hydrocarbon.

The invention is useful for mixing two or more fluid streams especially for streams, where considerable variations in flow rates occur and proper mixing is important.

An example, where thorough mixing is required, is the above mentioned CPO process. This process is an important process all over the world as H₂/CO synthesis gas is feed gas for numerous processes, of which some examples are hydrogen production, methanol production, formaldehyde production.

### EXAMPLES

One embodiment of the invention is described below. A mixer according to the invention often will be installed with the spindle in vertical position, which is assumed below.

The below described embodiment is a mixer of a size suitable for a pilot plant, for demonstrating a design of a commercial CPO reactor. The invention is not in any way limited to small sizes of reactors and mixers.

The mixer is 40-80 preferably 55-65 mm high and outer diameters are 40-80 preferably 55-65 mm.

The spindle is 100-400 preferably 200-300 mm long, the plug is 40-80 preferably 55-65 mm high and together with the seat it forms 1-5 preferably 2-4 annular spaces.

The space of the annuli are 0.25-1 preferably 0.6-0.7 mm. In a mixer with three annuli the three conical parts of the seat have min/max diameters 9.3-12.3 mm, 12.3-14.0 mm and 14.0-18.3 mm, respectively.

The conical surfaces form an angel of 10°-30° preferably 17.4°-17.6° with the axis of the spindle.

The chambers between the annuli have an outer diameter of 30-55 preferably 35-45 mm, and a height of 3-7 preferably 4-6 mm, and the holes forming the bore hole connections are 2-6 preferably 3-5 mm high with a diameter of 3-8 preferably 5-7 mm.

During the operation the spindle can move 5-10 preferably 6-8 mm up or down.

Inlet for gases to be mixed comprises 2-9 preferably 3-5 holes each with diameter 3-8 preferably 5-7 mm; and outlet for the mixed gas comprises 2-9 preferably 5-7 holes each with diameter 3-8 preferably 5-7 mm.

This embodiment is suitable for mixing a hydrocarbon with an oxidant, where the combined gaseous streams form a flow of 170-190 preferably 175-185 Nm³/h with a molecular weight of 2-50 preferably 21-23 gram/mole at 20-650 preferably 190-210° C., and the mixing takes place at 0.5-4.5 preferably 2.9-3.1 MPa.

Another embodiment of the invention is a mixer of industrial size.

This mixer is 400-800 preferably 550-650 mm high and outer diameters are 400-800 preferably 550-650 mm.

The spindle is 100-700 preferably 200-500 mm long, the plug is 400-800 preferably 550-650 mm high and together with the seat it forms 1-6 preferably 2-4 annular spaces.

The spaces of the annuli are 2.5-10 preferably 5.5-7.5 mm. The conical part of the seat has middle diameters 50-200 preferably 95-180 mm.

The conical surfaces form an angel of 10°-45° preferably 17.4°-17.6° with the axis of the spindle.

The chambers between the annuli have an outer diameter of 300-550 preferably 350-450 mm, and a height of 30-70 preferably 40-60 mm, and the holes are 20-60 preferably 30-50 mm high with a diameter of 30-80 preferably 50-70 mm.

During the operation the spindle can move 10-100 preferably 60-80 mm up or down.

Inlet for gases to be mixed comprises 2-9 preferably 3-5 holes, each with diameter 30-90 preferably 50-70 mm; and outlet for the mixed gas comprises 2-9 preferably 5-7 holes each with diameter 30-90 preferably 50-70 mm.

This embodiment is suitable for mixing a hydrocarbon with an oxidant, where the combined gaseous streams form a flow of 17000-19000 preferably 17500-18500 Nm³/h with a molecular weight of 2-50 preferably 21-23 gram/mole at 20-650 preferably 190-210° C., and the mixing takes place at 0.5-4.5 preferably 2.9-3.1 MPa.

One embodiment of the cage mixer is a mixer, where the body has an outer diameter of 60-65 preferably 61-63 mm, and 3-8 preferably 4-6 mixing elements are installed, each having a height of 5-15 preferably 8-12 mm, an outer diameter of 30-40 preferably 35-37 mm and an inner diameter of 18-22 preferably 19-21 mm. The cage inside the elements thus has a diameter of 18-22 preferably 19-21 mm and each element is equipped with 3-10 preferably 4-8 nozzles with a size of 1-5 preferably 1-3 mm. The nozzles are arranged in a helical pattern. Near the spindle the tube plug has a number of rows with holes, each row has 4-10, preferably 6-8, holes in a 6-14 preferably 8-12 mm square pitch and with a hole diameter of 3-7 preferably 4-6 mm.

The non-perforated part of the tube plug has a length/-height of 35-75 preferably 38-60 mm.

This embodiment is useful for a total gas stream of 170-190 preferably 175-185 Nm³/h with molecular weight 2-50 preferably 21-23 gram/mole at 0.5-4.5 preferably 2.9-3.1 MPa and 20-650 preferably 190-210° C.

1. A method for mixing two or more fluid streams
characterised in that
the streams are combined in an inlet chamber and thereafter repeatedly accelerated and decelerated in one or more stages, and
a value of a maximum linear velocity of the accelerated combined gaseous stream is maintained in each step within a range of mass flow rates of the streams.

2. A method according to claim 1, wherein the maximum linear velocity is maintained by adjusting area of smallest flow passage during a mixing operation.

3. A method according to claim 1, wherein additional mixing is obtained by 90 degree change in flow direction of the decelerated combined streams one or more times, preferably 3-5 times in each stage.

4. An apparatus for mixing two or more gaseous or liquid streams according to claim 1,
characterised in that it comprises
a body;
a seat;
a spindle with a plug;
an inlet chamber;
the plug installed inside the seat;
the seat and the plug have a plurality of conical surfaces forming the same plurality of conical annuli;
the seat is shaped to form two chambers between two annuli and with a bore flow connection between the said two chambers; and
the spindle is able to move the plug in an axial direction in the seat during the mixing operation.

5. An apparatus according to claim 4, wherein a porous medium is installed in the inlet chamber.

6. An apparatus according to claim 3, wherein a relationship between a gas flow rate and a cross sectional flow area of an annulus is expressed as
$${R}_{1} = \frac{F}{{D}_{seat}^{2} - {D}_{plug}^{2}} * \frac{{P}_{ref}}{P} * \frac{T}{{T}_{ref}}$$
where
F is total gas flow rate in Nm³/sec
Dₛₑₐₜ and D_{plug} are inner diameter of seat and diameter of plug at the same position in an annulus in m,
P is pressure in MPa in the mixer and P_{ref} is 3.0 MPa,
T is temperature in K in the mixer and T_{ref} is 473.15 K.
R₁ is in a range between 1*10⁶ and 1*10⁸ Nm³/sec/m² preferably between 5*10⁶ and 2*10⁷ Nm³/sec/m²; and
wherein a relation between the gas flow rate and a cross sectional flow area of holes forming the bore connection between the chambers is expressed as
$${R}_{2} = \frac{F}{n * {D}_{hole}^{2}} * \frac{{P}_{ref}}{P} * \frac{T}{{T}_{ref}}$$
where
F is total gas flow rate in Nm³/sec
n is number of holes,
Dₕₒₗₑ is diameter of holes between chambers in m,
P is pressure in MPa in the mixer and P_{ref} is 3.0 MPa,
T is temperature in K in the mixer and T_{ref} is 473.15 K; and
R₂ is in a range between 5*10⁵ and 1*10⁷ Nm³/sec/m² preferably 1*10⁶ and 2*10⁶ Nm³/sec/m².

7. An apparatus according to claims 4, wherein the spindle and plug further comprise an internal liquid flow passage and a spray nozzle connected to an outlet end of the internal liquid flow passage.

8. An apparatus for mixing two or more gaseous or liquid feed streams according to claim 1
characterised in that it comprises
a body;
a movable spindle connected to a tube plug coaxially installed in the body; where
the tube plug being perforated at an end adjacent to the spindle and open in other end; and
the feed streams enter the tube plug through perforation holes;
a cage with nozzles surrounding the tube plug and substantially without space from the tube plug; where
the height of the cage—when vertically installed—and the height of non-perforated part of the tube plug being substantially the same; and
the non-perforated part of the tube plug being able to block zero, some or all of the nozzles, when positioned in upper, a middle or lower position; and
a plurality of annular mixing elements surrounding the cage; where
the mixing elements being isolated from each other;
the cage and the mixing elements being closed at the end, where the tube plug end is open; and
total flow area of the cage nozzles is considerably smaller than flow area of any other flow passage of the mixer.

9. An apparatus according to claim 8, wherein a porous medium is installed upstream of the perforation holes and a porous medium is installed in the tube plug.

10. An apparatus according to claim 4, wherein
the feed streams are a hydrocarbon stream, a water vapour stream and an oxidant stream.

11. An apparatus according to claim 10, wherein the mixed stream form a feed stream for a catalytic partial oxidation process.
